(19) United States
(12) Patent Application Publication
(10) Pub. No.: US 2009/0137485 A1
(43) Pub. Date: May 28, 2009

(54) Antigens Targeted by Pathogenic T Cells in Type 1 Diabetes and Uses Thereof

(76) Inventors: Teresa P. Dilorenzo, Bayside, NY (US); Toshiyuki Takaki, Okayama-ken (JP); David V. Serreze, Ellsworth, ME (US); Michele Marron, Birmingham, AL (US)

Correspondence Address: AMSTER, ROTHSTEIN & EBENSTEIN LLP, 90 PARK AVENUE, NEW YORK, NY, 10016, US

(21) Appl. No.: 11/886,482

(22) PCT Filed: Mar. 9, 2006
(86) PCT No.: PCT/US06/09305

§ 371 Date: Sep. 3, 2008

Related U.S. Application Data
(60) Provisional application No. 60/662,737, filed on Mar. 17, 2005.

(51) Int. Cl. A61K 38/08; C07K 7/00; G01N 33/53

(52) U.S. Cl. 514/15; 530/328; 435/7.24

(57) ABSTRACT

Provided are polypeptides that are capable of binding a human HLA-A2 MHC class I molecule. Kits comprising these polypeptides in a container are also provided. Further provided are methods for determining whether a mammal is at risk for or has type 1 diabetes. Additionally provided are methods of preventing a CD8⁺ T cell that is cytotoxic to pancreatic islet β-cells from destroying a β-cell. Methods of treating a mammal that is at risk for type 1 diabetes are also provided, as are methods of treating a mammal that has type 1 diabetes.

### CROSS-REFERENCE TO RELATED APPLICATION

This application claims the benefit of U.S. Provisional Patent Application Ser. No. 60/662,737, filed Mar. 17, 2005.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grants No. DK52956, DK64315, DK46266, DK51090, DK59717, and DK69280, awarded by the National Institutes of Health.

### BACKGROUND OF THE INVENTION

(1) Field of the Invention

The present invention generally relates to diagnosis and therapy of type 1 diabetes. More specifically, the invention provides methods of diagnosis, prevention and therapy of type 1 diabetes based on the identification of islet β cell antigen epitopes targeted by pathogenic T cells restricted to the human MHC class I molecule HLA-A2.

(2) Description of the Related Art

### REFERENCES CITED

Airman, J. D. et al. 1996. Science 274:94.
Amrani, A., P. Serra, J. Yamanouchi, J. D. Trudeau, R. Tan, J. F. Elliott, and P. Santamaria. 2001. Expansion of the antigenic repertoire of a single T cell receptor upon T cell activation. J. Immunol. 167:655-666.
Choisy-Rossi, C.-M., T. M. Holl, M. A. Pierce, H. D. Chapman, and D. V. Serreze. 2004. Enhanced pathogenicity of diabetogenic T cells escaping a non-MHC gene-controlled near death experience. J Immunol 173:3791.
Christianson, S. W., L. D. Shultz, and E. H. Leiter. 1993. Adoptive transfer of diabetes into immunodeficient NOD-scid/scid mice. Relative contributions of CD4⁺ and CD8⁺ T-cells from diabetic versus prediabetic NOD.NON-Thy-1^{a} donors. Diabetes 42:44.
DiLorenzo, T. P., R. T. Graser, T. Ono, G. J. Christianson, H. D. Chapman, D. C. Roopenian, S. G. Nathenson, and D. V. Serreze. 1998. Major histocompatibility complex class I-restricted T cells are required for all but the end stages of diabetes development in nonobese diabetic mice and use a prevalent T cell receptor a chain gene rearrangement. Proc. Natl. Acad. Sci. U.S.A. 95:12538.
DiLorenzo, T. P., S. M. Lieberman, T. Takalci, S. Honda, H. D. Chapman, P. Santamaria, D. V. Serreze, and S. G. Nathenson. 2002. During the early prediabetic period in NOD mice, the pathogenic CD8⁺ T-cell population comprises multiple antigenic specificities. Clin. Immunol. 105:332.
Gammon et al. 1986. Nature (London) 319:413.
Graser, R. T., T. P. DiLorenzo, F. Wang, G. J. Christianson, H. D. Chapman, D. C. Roopenian, S. G. Nathenson, and D. V. Serreze. 2000. Identification of a CD8 T cell that can independently mediate autoimmune diabetes development in the complete absence of CD4 T cell helper functions. J. Immunol. 164:3913.
Gurlo, T., K. Kawamura, and H. von Grafenstein. 1999. Role of inflammatory infiltrate in activation and effector function of cloned islet reactive nonobese diabetic CD8⁺ T cells: involvement of a nitric oxide-dependent pathway. J. Immunol. 163:5770-5780.
Hartemann, A. H. et al. 1999. Clin, Exper. Immunol. 116:225.
Karttunen, J., S. Sanderson, and N. Shastri. 1992. Detection of rare antigen-presenting cells by the lacZ T-cell activation assay suggests an expression cloning strategy for T-cell antigens. Proc Natl Acad Sci USA 89:6020.
Leiter, E. H. 1997. The NOD mouse: A model for insulin-dependent diabetes mellitus. In Current Protocols in Immunology. J. E. Coligan, A. M. Kruisbeek, D. H. Margulies, E. M. Shevach, and W. Strober, eds. John Wiley & Sons, Inc., Hoboken, p. 15.9.1.
Lieberman, S. M., and T. P. DiLorenzo. 2003. A comprehensive guide to antibody and T-cell responses in type 1 diabetes. Tissue Antigens 62:359.
Lieberman, S. M., T. Takaki, B. Han, P. Santamaria, D. V. Serreze, and T. P. DiLorenzo. 2004. Individual NOD mice exhibit unique patterns of CD8⁺ T cell reactivity to three islet antigens, including the newly identified widely expressed dystrophia myotonica kinase. J. Immunol. 173:6727-6734.
Nagata, M., P. Santamaria, T. Kawamura, T. Utsugi, and J. W. Yoon. 1994. Evidence for the role of CD8⁺ cytotoxic T cells in the destruction of pancreatic β-cells in nonobese diabetic mice. J. Immunol. 152:2042-2050.
Pascolo, S., N. Bervas, J. M. Ure, A. G. Smith, F. A. Lemonnier, and B. Perarnau. 1997. HLA-A2.1-restricted education and cytolytic activity of CD8⁺ T lymphocytes from β2 microglobulin (β2m) HLA-A2.1 monochain transgenic H-2D^{b} β2m double knockout mice. J Exp Med 185:2043.
Panagiotopoulos, C., H. Qin, R. Tan, and C. B. Verchere. 2003. Identification of a β-cell-specific HLA class I restricted epitope in type 1 diabetes. Diabetes 52:2647
Regner, M., M. H. Claesson, S. Bregenholt, and M. Ropke. 1996. An improved method for the detection of peptide-induced upregulation of HLA-A2 molecules on TAP-deficient T2 cells. Exp Clin Immunogenet 13:30.
Rodda, S. J. 2002. Peptide libraries for T cell epitope screening and characterization. J Immunol Methods 267:71.
Roep, B. O. 2003. The role of T-cells in the pathogenesis of Type 1 diabetes: from cause to cure. Diabetologia 46:305-321.
Russo, C, A. K. Ng, M. A. Pellegrino, and S. Ferrone. 1983. The monoclonal antibody CR11-351 discriminates HLA-A2 variants identified by T cells. Immunogenetics 18:23.
Santamaria, P., T. Utsugi, B. J. Park, N. Averill, S. Kawazu, and J. W. Yoon. 1995. Beta-cell-cytotoxic CD8⁺ T cells from nonobese diabetic mice use highly homologous T cell receptor α-chain CDR3 sequences. J. Immunol. 154:2494-2503.
Serreze, D. V., and E. H. Leiter. 2001a. Genes and pathways underlying autoimmune diabetes in NOD mice. In Molecular Pathology of Insulin Dependent Diabetes Mellitus. M. G. von Herrath, ed. Karger Press, New York, p. 31.
Serreze, D. V., and E. H. Leiter. 2001b. Genes and cellular requirements for autoimmune diabetes susceptibility in nonobese diabetic mice. Curr. Dir. Autoimmun. 4:31-67.
Serreze, D. V., E. H. Leiter, G. J. Christianson, D. Greiner, and D. C. Roopenian. 1994. MHC class I-deficient NOD-B2m^{null} mice are diabetes and insulitis resistant. Diabetes 43:505.
Shultz, L. D., P. A. Lang, S. W. Christianson, B. Gott, B. Lyons, S. Umeda, E. Leiter, R. Hesselton, E. J. Wagar, J. H. Leif, O. Kollet, T. Lapidot, and D. L. Greiner. 2000. NOD/LtSz-Rag1^{null} mice: An immunodeficient and radioresistant model for engraftment of human hematolymphoid cells, HIV infection, and adoptive transfer of NOD mouse diabetogenic T cells. J Immunol 164:2496.
Takaki, T., S. M. Lieberman, T. M. Holl, B. Han, P. Santamaria, D. V. Serreze, and T. P. DiLorenzo. 2004. Requirement for both H-2D^{b} and H-2K^{d} for the induction of diabetes by the promiscuous CD8⁺ T cell clonotype AI4. J. Immunol. 173:2530-2541.
Trudeau, J. D., C. Kelly-Smith, C. B. Verchere, J. F. Elliott, J. P. Dutz, D. T. Finegood, P. Santamaria, and R. Tan. 2003. Prediction of spontaneous autoimmune diabetes in NOD mice by quantification of autoreactive T cells in peripheral blood. J. Clin. Invest. 111:217.
Utsugi, T., J. W. Yoon, B. J. Park, M. Imamura, N. Averill, S. Kawazu, and P. Santamaria. 1996. Major histocompatibility complex class I-restricted infiltration and destruction of pancreatic islets by NOD mouse-derived β-cell cytotoxic CD8⁺ T-cell clones in vivo. Diabetes 45:1121-1131.
Verdaguer, J., D. Schmidt, A. Armani, B. Anderson, N. Averill, and P. Santamaria. 1997. Spontaneous autoimmune diabetes in monoclonal T cell nonobese diabetic mice. J. Exp. Med. 186:1663.
Wang, B., A. Gonzalez, C. Benoist, and D. Mathis. 1996. The role of CD8⁺ T cells in the initiation of insulin-dependent diabetes mellitus. Eur. J. Immunol. 26:1762-1769.
Wicker, L. S., E. H. Leiter, J. A. Todd, R. J. Renjilian, E. Peterson, P. A. Fischer, P. L. Podolin, M. Zijlstra, R. Jaenisch, and L. B. Peterson. 1994. β₂-microglobulin-deficient NOD mice do not develop insulitis or diabetes. Diabetes 43:500.
Wong, F. S., I. Visintin, L. Wen, R. A. Flavell, and C. A. Janeway, Jr. 1996. CD8 T cell clones from young nonobese diabetic (NOD) islets can transfer rapid onset of diabetes in NOD mice in the absence of CD4 cells. J. Exp. Med. 183:67.
Wong, F. S., J. Karttunen, C. Dumont, L. Wen, I. Visintin, I. M. Pilip, N. Shastri, E. G. Pamer, and C. A. Janeway, Jr. 1999. Identification of an MHC class I-restricted autoantigen in type 1 diabetes by screening an organ-specific cDNA library. Nat. Med. 5:1026.
PCT Patent Application Publication WO05/033257.
PCT Patent Application Publication WO06/023211.

In both humans and NOD mice, type 1 diabetes (T1D) is an autoimmune disease that results from T cell-mediated destruction of insulin-producing pancreatic β cells and involves complex interactions among developmental, genetic, and environmental factors (Serreze and Leiter, 2001a; 2001b; Roep, 2003). In the NOD mouse model, spontaneous autoimmune diabetes development requires both CD4⁺ and CD8⁺ T cells (Christianson et al., 1993; Serreze et al., 1994; Wicker et al., 1994; Wang et al., 1996; DiLorenzo et al., 1998), with evidence suggesting that CD8⁺ T cells are required for the initial stages of p cell destruction (Wang et al., 1996; DiLorenzo et al., 1998). Although there are multiple susceptibility loci, the strong association of particular MHC class II molecules with disease has led to extensive investigation of CD4⁺ T cells in T1D (Lieberman and DiLorenzo, 2003). However, several studies in NOD mice have documented the importance of pathogenic CD8⁺ T cells in the initial stages of β cell destruction (DiLorenzo et al., 1998, Serreze et al., 1994; Wong et al., 1996; Wicker et al, 1994).

Several NOD-derived, β cell-autoreactive CD8⁺ T cell clones have been reported (DiLorenzo et al., 1998; 2002; Gurlo et al., 1999; Nagata et al., 1994; Wong et al., 1996; Shimizu et al., 1993); however, only three of these (designated G9C8, 8.3, and AI4) have demonstrated in vivo pathogenicity.

The 8.3 clone represents a prevalent population of islet-specific glucose-6-phosphatase catalytic subunit-related protein₂₀₆₋₂₁₄ (IGRP₂₀₆₋₂₁₄)-reactive T cells present in NOD islets throughout disease development and progression to overt diabetes (DiLorenzo et al., 1998; Santamaria et al., 1995; Lieberman et al., 2003; Amrani et al., 2000; Trudeau et al., 2003; PCT Patent Application Publication WO05/033257, incorporated by reference). The pathogenicity of 8.3 is demonstrated by the accelerated rate of diabetes development observed in 8.3 TCR transgenic NOD mice that is enhanced by CD4⁺ T cell help (Verdaguer et al., 1997), and by adoptive transfer studies (Nagata et al., 1994; Utsugi et al., 1996).

Other IGRP peptides have also been identified that react to islet-derived CD8⁺ T-cells from NOD mice (WO05/033257).

The insulin B₁₅₋₂₃-reactive pathogenic CD8⁺ T cell clone G9C8 has been shown to cause diabetes in the absence of CD4⁺ T cell help, but these experiments involved transfer of previously activated G9C8 T cells into recipient mice; thus, their ability to develop and mature in the absence of CD4⁺ T cell help is unknown (Wong et al., 1996; 1999).

The AI4 CD8⁺ T cell clone, originally isolated from the islets of a 5-week-old female NOD mouse (DiLorenzo et al., 1998), represents another p cell-autoreactive specificity. NOD mice transgenically expressing the AI4 TCR (designated NOD.AI4αβ Tg) progress to overt diabetes significantly earlier than nontransgenic NOD mice (Graser et al., 2000). Antigens reacting to the AI4 CD8⁺ T cell clone have also been recently identified (PCT Patent Application Publication WO06/023211, Takaki et al., 2004; Lieberman et al., 2004).

It would be desirable to identify epitopes reactive to pathogenic T cells expressing a human MHC class I molecule. The present invention addresses that need.

### SUMMARY OF THE INVENTION

Accordingly, the inventors have identified peptides from islet-specific glucose-6-phosphatase catalytic subunit-related protein (IGRP) that react with HLA-A2-restricted T cells from transgenic NOD mice.

Thus, in some embodiments, the invention is directed to isolated and purified polypeptides comprising at least one of amino acid sequences (F/L)(G/N)IDLLWSV, VLFGLGFAI, and A(F/L)IPY(C/S)VHM. In these embodiments, the polypeptide does not comprise any of the sequences YLKTN(A/I/L/V)FL, FLWSVFWLI, (T/A)YY(G/T)FLNFM, LR(L/V)(F/L)(G/N)IDLL, KWCANPDWI, or SFCKSASIP.

The invention is also directed to isolated and purified polypeptides 8-10 amino acids in length, completely homologous with a mammalian IGRP having at least 90% homology to SEQ ID NO:1 or SEQ ID NO:2. These polypeptides are capable of binding a human HLA-A2 MHC class I molecule.

In additional embodiments, the invention is directed to methods for determining whether a mammal is at risk for or has type 1 diabetes. The methods comprise determining the presence of CD8⁺ T cells reactive to IGRP in the mammal by
a. obtaining a sample of lymphocytes comprising CD8⁺ T cells from the mammal;
b. combining the lymphocytes with a polypeptide and an MHC class I molecule that is capable of binding the polypeptide, where the polypeptide or the MHC molecule further comprises a detectable label and wherein the polypeptide is 8-10 amino acids in length and comprises one of the sequences (F/L)(G/N)H)LLWSV, VLFGLGFAI, or A(F/L)IPY(C/S)VHM; and
c. determining whether any CD8⁺ T cells specifically bind to the polypeptide. In these methods, CD8⁺ T cell binding to the polypeptide indicates that the mammal is at risk for or has type 1 diabetes.

In further embodiments, the invention is directed to additional methods for determining whether a mammal is at risk for or has type 1 diabetes. The methods comprise determining the presence of CD8⁺ T cells reactive to IGRP in the mammal by
a. obtaining a sample of lymphocytes comprising CD8⁺ T cells from the mammal;
b. combining the lymphocytes with
   i. a polypeptide capable of binding an MHC class I molecule HLA-A2 and
   ii. the MHC class I molecule HLA-A2, where the polypeptide or the HLA-A2 further comprises a detectable label and where the polypeptide is 8-10 amino acids and is completely homologous with a mammalian IGRP; and
c. determining whether any CD8⁺ T cells specifically bind to the polypeptide. In these embodiments, CD8⁺ T cell binding to the polypeptide indicates that the mammal is at risk for or has type 1 diabetes.

The invention is additionally directed to methods of preventing a CD8⁺ T cell that is cytotoxic to pancreatic islet β-cells from destroying a β-cell. The methods comprise treating the β-cell with a compound capable of specifically binding a polypeptide 8-10 amino acids in length that is completely homologous with a mammalian IGRP having at least 90% homology to SEQ ID NO:1 or SEQ ID NO:2, where the polypeptide is capable of binding a human HLA-A2 MHC class I molecule.

The invention is also directed to additional methods of preventing a CD8⁺ T cell that is cytotoxic to pancreatic islet (3-cells from destroying a mammalian β-cell. The methods comprise treating the CD8⁺ T cell with a polypeptide 8-10 amino acids in length in a manner sufficient to reduce CD8⁺ T cells reactive to IGRP, where the polypeptide comprises one of the sequences (F/L)(G/N)IDLLWSV, VDFGLGFAI, or A(F/L)IPY(C/S)VHM.

In further embodiments, the invention is directed to methods of treating a mammal that is at risk for type 1 diabetes. The methods comprise administering a polypeptide to the mammal in a manner sufficient to reduce CD8⁺ T cells reactive to IGRP, where the polypeptide is 8-10 amino acids in length, and comprises (F/L)(G/N)IDLLWSV, VLFGLGFAI, or A(F/L)IPY(C/S)VHM.

In related embodiments, the invention is also directed to additional methods of treating a mammal that is at risk for type 1 diabetes. The methods comprise administering a polypeptide to the mammal in a manner sufficient to reduce CD8⁺ T cells reactive to IGRP. In these embodiments, the polypeptide is 8-10 amino acids in length, completely homologous with a mammalian IGRP having at least 90% homology to SEQ ID NO:1 or SEQ ID NO:2, and is capable of binding a human HLA-A2 MHC class I molecule.

The present invention is also directed to methods of treating a mammal that has type 1 diabetes. The methods comprise administering a polypeptide to the mammal in a manner sufficient to treat diabetes, where the polypeptide is 8-10 amino acids in length, and comprises (F/L)(G/N)IDLLWSV, VLFGLGFAI, or A(F/L)IPY(C/S)VHM.

In related embodiments, the invention is directed to additional methods of treating a mammal that has type 1 diabetes. The methods comprise administering a polypeptide to the mammal in a manner sufficient to treat type 1 diabetes. In these methods, the polypeptide is 8-10 amino acids in length, completely homologous with a mammalian IGRP having at least 90% homology to SEQ ID NO: 1 or SEQ ID NO:2, and is capable of binding a human HLA-A2 MHC class I molecule.

Additionally, the invention is directed to kits comprising a sterile preparation of any of the above-described polypeptides, in a container.

### BRIEF DESCRIPTION OF THE DRAWINGS

FIG. 1 are graphs showing that transgenically expressed chimeric HLA-A2.1 MHC class I molecules contribute to development of type 1 diabetes in NOD mice. Female mice were monitored weekly for development of diabetes. Panel a. NOD.HHD mice (n=19, ▪) had significantly accelerated onset of disease (p=0.02) compared to non-transgenic littermates (n=27, ∘). Panel b. NOD.β2m^{null}.HHD mice (n=20, ▪) transgenically expressing HLA-A2.1 molecules were susceptible to diabetes development while non-transgenic NOD.β2m^{null} littermates (n=10, ∘), which lack MHC class I molecules, were completely resistant (p=0.006).

FIG. 2 is a graph showing that cultured islet-infiltrating T cells from NOD.β2m^{null}.HHD mice recognize murine IGRP peptides. Islets from five 12-wk-old female NOD.HHD mice (Panel a), four 10-wk-old female NOD.β2m^{null}.HHD mice (Panel b), or four 11-wk-old female NOD.β2m^{null}.HHD mice (Panel c) were pooled to obtain sufficient numbers of cells to screen the murine IGRP peptide library. Islets were cultured in the presence of IL-2, and islet-infiltrating T cells were harvested after 7 days. T2 cells were used as APC and T cell response was measured by IFN-γ ELISpot assay.

FIG. 3 shows the identification of three antigenic HLA-A2-binding peptides from murine IGRP. Panel a. Schematic of three positive peptide mixtures (229, 266, and 338) from the murine IGRP peptide library. Bold letters indicate anchor or auxiliary anchor residues based on HLA-A*0201 peptide motifs. Panel b. Individual peptides were synthesized, and their T cell stimulation ability was compared with the original peptide mixtures. Each graph shows a separate experiment using islet-infiltrating T cells cultured from individual 12-wk-old female NOD.β2m^{null}.HHD mice. Solid bars, peptide mixtures. Hatched bars, individual peptides. Panel c, T2 cells (HLA-A*0201) or RMA-S/K^{d} cells (H-2D^{b}, K^{b}, and K^{d}) were used as APC to confirm the HLA-A2-restriction of the IGRP-reactive T cells. Panel d. COS-7 cells were transiently transfected with varying concentrations of a murine IGRP expression construct and 1 μg/ml of an HHD (), D^{b} (x), or K^{d} (□) expression construct. Cultured islet-infiltrating T cells from 12-wk-old female NOD.β2m^{null}.HHD mice were added, and T cell response was measured as IFN-γ release by ELISA assay. T cells did not respond to COS-7 cells co-transfected with the murine IGRP expression construct and empty vector, nor did they respond to COS-7 cells co-transfected with the HHD expression construct and empty vector.

FIG. 4 shows the characterization of the human counterparts of the HLA-A2-binding murine IGRP peptides. Panel a. Schematic comparison of three HLA-A2-binding murine IGRP peptides with the corresponding peptides from human IGRP. Bold and underlined letters indicate amino acid differences between murine and human IGRP peptides. Panel b. HLA-A2 stabilization assay for the three HLA-A2-binding murine IGRP peptides and their human counterparts. T2 cells were pulsed with the indicated peptides, stained with FITC-conjugated anti-HLA-A2 mAb BB7.2, and analyzed by flow cytometry. Each bar represents fluorescence index calculated as described in METHODS. PreproIAPP₅₋₁₃ represents an HLA-A2-binding human islet amyloid polypeptide (LAPP) precursor peptide to which T cells from recent-onset type 1 diabetes patients have been reported to respond (Panagiotopoulos et al., 2003). Flu MP₅₈₋₆₆ is an HLA-A2-binding influenza matrix protein (MP) peptide used as a positive control (Regner et al, 1996). Panel c. Murine and human IGRP peptides (228-236 and 337-345) were compared regarding their ability to stimulate cultured islet-infiltrating T cells from an NOD.β2m^{null}.HHD mouse using IFN-γ ELISpot assay.

FIG. 5 shows that individual NOD.β2m^{null}.HHD mice exhibit distinct patterns of CD8⁺ T cell reactivity to three HLA-A2-binding murine IGRP peptides. Panel a. Islets were isolated from ten 12-wk-old female NOD.β2m^{null}.HHD mice and cultured individually in the presence of IL-2 for 7 days. Response of the resulting T cells to the three peptides was measured by IFN-γ ELISpot assay. Panel b. Summary of quantification of ELISpot assays performed on cultured islet-infiltrating T cells from 16 individual 12 to 13-wk-old female NOD.β2m^{null}.HHD mice.

FIG. 6 is a graph of experimental results showing that cultured islet-infiltrating T cells from NOD.β2m^{null}.HHD mice do not show cytotoxic activity against HLA-A2-positive or HLA-A2-negative human peripheral blood mononuclear cells (PBMCs). Peripheral blood was collected from both HLA-A2-positive and HLA-A2-negative subjects. PBMCs were isolated by using Ficoll-Paque™ PLUS (GE Healthcare), and 3×10⁶ PBMCs were cultured with 2 μg/ml Concanavalin A (Con A, Sigma-Aldrich) for 2 days in a 37° C. CO₂ incubator for use as target cells. Islets were isolated from 12-wk-old female NOD.β2m^{null}.HHD mice and islet-infiltrating T cells were cultured in the presence of IL-2 for 7 days. Cytotoxic activities of islet-infiltrating T cells were measured by ⁵¹Cr release assay.

FIG. 7 is a graph of experimental results showing that CD8⁺ T cells from early insulitic lesions of NOD.β2m^{null}.HHD mice are reactive to three HLA-A2.1-binding murine IGRP peptides. Islets were isolated from seven 5-wk-old female NOD.β2m^{null}.HHD mice and cultured individually in the presence of IL-2 for 7 days. The response of the resulting T cells to the three peptides was measured by IFN-γ ELISPOT assay using T2 cells as antigen-presenting cells. Responses in the absence of peptide have been subtracted. Thus, all responses shown are peptide-specific.

### DETAILED DESCRIPTION OF THE INVENTION

Accordingly, the inventors have identified peptides from islet-specific glucose-6-phosphatase catalytic subunit-related protein (IGRP) that react with HLA-A2-restricted T cells from transgenic NOD mice. The peptides are (F/L)(G/N)IDLLWSV, VLFGLGFAI, and A(F/L)IPY(C/S)VHM, where each letter is an amino acid residue using the common one letter amino acid code, and where two letters in parenthesis, separated by a slash, indicates either of the two amino acids can be at that position in the sequence.

As used herein, a “peptide” or “polypeptide” is a linear sequence of at least two amino acids, or a peptidomimetic thereof. As used herein, a peptidomimetic is a chain of at least two amino acids or amino acid analogs, where there is at least one amino acid analog, and where the peptidomimetic has the same T cell binding characteristics as the corresponding polypeptide consisting exclusively of amino acids.

Of the three IGRP peptides, (F/L)(G/N)IDLLWSV is at position 228-236 of IGRP, where the first-listed residue at the variable positions is the mouse residue at that position and the second-listed residue is the human residue. The sequence VLFGLGFAI is at IGRP position 265-273, and A(F/L)IPY(C/S)VHM is at IGRP position 337-345.

Thus, in some embodiments, the invention is directed to isolated and purified polypeptides comprising at least one of amino acid or analog sequences (F/L)(G/N)IDLLWSV, VLFGLGFAI, and A(F/L)IPY(C/S)VHM. In these embodiments, the polypeptide does not comprise any of the sequences YLKTN(A/I/L/V)FL, FLWSVFWLI, (T/A)YY(G/T)FLNFM, LR(L/V)(F/L)(G/N)IDLL, KWCANPDWI, or SFCKSASLP. The latter excluded sequences are IGRP peptides that were already found to be useful. See PCT Patent Application Publication WO05/033257.

As used herein, an IGRP amino acid sequence includes any naturally occurring mammalian amino acid sequence that is islet specific and is at least 90% identical to SEQ ID NO:1 (Mouse IGRP, from GenBank NP 067306) or SEQ ID NO:2 (Human IGRP, from GenBank NP 066999). The IGRP mouse and human amino acid sequences are not limited to SEQ ID NO:1 and SEQ ID NO:2, respectively, but includes any variants naturally present in mice or humans. The identification of any mammalian IGRP amino acid sequence can be readily made without undue experimentation, e.g., by identifying mRNA sequences limited to islet cells that are highly (i.e., >90%) homologous to already-identified mRNA sequences of mouse or human IGRP (e.g., as found in GenBank accessions NP 021331 and NP 021176, respectively), and determining the amino acid sequence of the expressed protein. Thus, in preferred embodiments, the polypeptide is completely homologous to a mammalian IGRP having at least 90% homology to SEQ ID NO:1 or SEQ ED NO:2.

As used herein, “isolated and purified” means present in a greater concentration than would be found in nature. Preferably, an isolated and purified polypeptide is at least about 10% of the peptide component of the preparation; more preferably at least about 25%; even more preferably at least about 50%; still more preferably at least about 75%; and most preferably at least about 90% of the peptide component of a preparation.

In other preferred embodiments, the polypeptides comprise less than about 100 amino acids or ammo acid analogs, more preferably less than about 25 amino acids or amino acid analogs, even more preferably 13-25 amino acids or analogs, and most preferably 8-10 amino acids or analogts, since CD8⁺ T cells generally only bind polypeptides of 8-10 amino acids. In these embodiments, preferred polypeptides comprise the above peptides having the sequence FGDDLLWSV, LNTOLLWSV, VLFGLGFAI, ALIPYCVHM, or AFJPYSVHM, since those sequences are present in the naturally occurring IGRP from mice and humans.

In further aspects of these embodiments, the polypeptide also comprises an antigenic earner, in order to more effectively use the peptides in immunization protocols, to tolerize a mammal to IGRP, preventing development of type 1 diabetes, as was achieved in the experiment described in WO05/033257. A nonlimiting example of an antigenic carrier is incomplete Freund's adjuvant. See also Gammon et al., 1986.

In other aspects, the polypeptide further comprises a detectable label. Such labeled peptides are useful in diagnostic protocols, e.g., to determine the presence of CD8⁺ T cells reactive to the peptides, to identify a mammal that has, or is at risk for, type 1 diabetes. The invention is not limited to any particular detectable label, and the skilled artisan can select a label most appropriate for any particular application without undue experimentation. Examples include a fluorescent moiety, a radioactive molecule, and an assayable enzyme (e.g., β-galactosidase or streptavidin). Methods for labeling peptides with any of these detectable moieties are well known.

In further aspects, the above-identified polypeptides of 8-10 amino acids or analogs can be usefully combined with an MHC class I molecule that is capable of binding the polypeptide, preferably a human HLA-A2 molecule, which binds the polypeptides. Since CD8⁺ T cells only bind to an antigen in the context of an MHC class I molecule, the polypeptide-MHC class I mixtures are useful for creating a T cell ligand. The MHC class I molecules are preferably employed in the form of tetramers. See, e.g., Altaian et al., 1996; Trudeau et al., 2003. In some applications, e.g., diagnostics, the polypeptide of the polypeptide-MHC class I mixtures further comprises a detectable label, such as those discussed above, i.e., conjugated to the polypeptide. Alternatively or additionally, the MHC class I molecule could employ a detectable label.

In some methods of treatment, directed toward eliminating CD8⁺ T cells, the polypeptide or the MHC class I molecule can also include a cytotoxic molecule. In these methods, the cytotoxic polypeptide-MHC class I mixture binds to the CD8⁺ T cells reactive to the IGRP epitopes corresponding to the polypeptide, where the cytotoxic molecule kills the T cell.

The invention is not narrowly limited to any particular cytotoxic molecule that is bound to the polypeptide or MHC class I molecule. The skilled artisan could identify various cytotoxic molecules useful in these aspects, and could select the appropriate cytotoxic molecule for any particular application without undue experimentation. Examples of potentially useful cytotoxic molecules include radioactive molecules (e.g., ¹³¹I, ⁹⁰Y), and toxic chemicals or proteins (e.g., 5-fluorouridine or ricin).

Since CD4⁺ T cells are also involved in the pathogenic process of type 1 diabetes, and since CD4⁺ T cells bind polypeptides that are 13-25 amino acids, and only when presented on MHC class II molecules, mixtures of polypeptides with MHC class II molecules are also within the scope of the invention. In these embodiments, the polypeptides are 13-25 amino acids or analogs and comprise one of the sequences (F/L)(G/N)IDLLWSV, VLFGLGFAI, or A(F/L)IPY(C/S)VHM.

Analogous to previously described polypeptide-MHC class I mixtures, the polypeptides or MHC class II molecules of the polypeptide-MHC class II mixtures can also usefully comprise a detectable label or a cytotoxic molecule.

It is also envisioned that any of the above-described polypeptides are usefully provided in a sterile pharmaceutical preparation, particularly when the polypeptide is to be utilized for therapeutic treatments. Thus, in some embodiments, the polypeptide in a sterile pharmaceutical preparation is capable of tolerizing a mammal to reduce CD8⁺ T cells reactive to IGRP. Preferably, the polypeptide comprises at least one of the sequences FGIDLLWSV, LNIDLLWSV, VLFGLGFAI, ALEPYCVHM, or AFEPYSVHM.

In other embodiments, the invention is directed to isolated and purified polypeptides 8-10 amino acids or analogs in length that are completely homologous with a mammalian IGRP having at least 90% homology to SEQ ID NO:1 or SEQ ID NO:2. In these embodiments, the polypeptides are capable of binding a human HLA-A2 MHC class I molecule. Preferably, the polypeptides comprise one of the amino acid or analog sequences (F/L)(G/N)IDLLWSV, VLFGLGFAI, or A(F/L)IPY(C/S)VHM. More preferably, the polypeptides of these embodiments comprise at least one of the sequences FGIDLLWSV, LNIDLLWSV, VLFGLGFAI, ALEPYCVHM, or AFJPYSVHM.

Analogous to the embodiments described above, these polypeptides can also comprise a detectable label, such as a fluorescent molecule, a radioactive molecule, or an enzyme. The polypeptide can also further comprise a human HLA-A2 MHC class I molecule, which binds the polypeptides. In additional embodiments, the polypeptides can also comprise a cytotoxic molecule, e.g., a radioactive molecule.

In some preferred embodiments, the invention is directed to isolated and purified polypeptides consisting of 8-10 amino acids or analogs and comprising the sequence FGIDLLWSV, LNIDLLWSV, VLFGLGFAI, ALLPYCVHM or AFLPYSVHM.

The above-described polypeptides can be used diagnostically to determine whether a mammal is at risk for or has type 1 diabetes. Thus, the present invention is also directed to methods for determining whether a mammal is at risk for or has type 1 diabetes. The methods comprise determining the presence of CD8⁺ T cells reactive to IGRP in the mammal by
a. obtaining a sample of lymphocytes comprising CD8⁺ T cells from the mammal by standard methods (e.g., venipuncture);
b. combining the lymphocytes with a polypeptide and an MHC class I molecule that is capable of binding the polypeptide, where the polypeptide or the MHC molecule further comprises a detectable label and where the polypeptide is 8-10 amino acids or analogs in length and comprises one of the sequences (F/L)(G/N)IDLLWSV, VLFGLGFAI, or A(F/L)IPY(C/S)VHM; and
c. determining whether any CD8⁺ T cells specifically bind to the polypeptide, where CD8⁺ T cell binding to the polypeptide indicates that the mammal is at risk for or has type 1 diabetes. Preferably, the polypeptide comprises one of the sequences FGIDLLWSV, LNIDLLWSV, VLFGLGFAI, ALTRYCVHM, or AFIPYSVHM.

These methods can be used with any mammal, although the mammal is preferably a mouse or a human.

The determination step (c.) can be by any known means. In some preferred embodiments, the determination step is performed by counting labeled CD8⁺ T cells using a cell sorter (e.g., a fluorescence activated cell sorter) or labeled cell counter (e.g., Coulter counter). These embodiments allow quantification of the amount of peptide binding, and can thus be used to estimate relative risk, or severity of disease. In other preferred embodiments, the determination step is performed by microscopic observation of the lymphocytes under conditions where the label can be observed, e.g., with a fluorescence microscope if a fluorescent label is used, or light microscope if an enzyme label and colored enzyme substrate is used to visualize the bound T cells. In additional preferred embodiments, the determination step is performed by measuring activation of the CD8⁺ T cells, preferably by measuring interferon-γ production by known methods, for example using an ELISpot assay (see, e.g., Hartemann et al., 1999).

The invention is also directed to additional methods for determining whether a mammal is at risk for or has type 1 diabetes. The methods comprise determining the presence of CD8⁺ T cells reactive to IGRP in the mammal by
a. obtaining a sample of lymphocytes comprising CD8⁺ T cells from the mammal;
b. combining the lymphocytes with
   i. a polypeptide capable of binding an MHC class I molecule HLA-A2 and
   ii. the MHC class I molecule HLA-A2, where the polypeptide or the HLA-A2 further comprises a detectable label and where the polypeptide is 8-10 amino acids or analogs and is completely homologous with a mammalian IGRP; and
c. determining whether any CD8⁺ T cells specifically bind to the polypeptide, where CD8⁺ T cell binding to the polypeptide indicates that the mammal is at risk for or has type 1 diabetes.

Preferably in these embodiments, the polypeptide comprises one of the sequences (F/L)(G/N)IDLLWSV, VLFGLGFAI, or A(F/L)IPY(C/S)VHM; more preferably one of the sequences FGIDLLWSV, LNIDLLWSV, VLFGLGFAI, ALIPYCVHM, or AFIPYSVHM.

Many of the above-described compositions are useful in methods of treating mammals (including but not limited to humans and rodents such as mice) that are at risk for, or have type 1 diabetes. As such, the above-described compositions can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for die adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

Thus, in further embodiments, the invention is directed to methods of preventing a CD8⁺ T cell that is cytotoxic to pancreatic islet β-cells from destroying a β-cell. The methods comprise treating the β-cell with a compound capable of specifically binding a polypeptide, where the polypeptide is 8-10 amino acids or analogs in length, completely homologous with a mammalian IGRP having at least 90% homology to SEQ ID NO:1 or SEQ ID NO:2, and is capable of binding a human HLA-A2 MHC class I molecule. These polypeptides are described above. By binding domains on β-cell IGRP, reaction of CD8⁺ T cells with IGRP, which can lead to the destruction of the β-cell, can be prevented. Preferably, the polypeptide comprises one of the sequences (F/L)(G/N)IDLLWSV, VLFGLGFAI, or A(F/L)IPY(C/S)VHM, most preferably FGIDLLWSV, LNIDLLWSV, VLFGLGFAI, ALEPYCVHM, or AFLPYSVHM.

In these embodiments, the compound capable of binding the polypeptide can be any compound capable of interfering with the CD8⁺ binding to the β cell, either by reducing the numbers of polypeptide-MHC binding, or by causing a physical interference to the T cell binding to the polypeptide-MHC complex. In preferred embodiments, the compound is an antibody or an aptamer.

Methods of making antibodies to a polypeptide are routine, and the skilled artisan would expect that such an antibody could be made to any IGRP polypeptide without undue experimentation. The antibodies can be from a polyclonal, monoclonal, or recombinant source. As used herein, “antibodies” also include a fragment of a whole antibody that comprises a typical immunoglobulin antigen binding site (e.g., Fab or Fab2). The antibodies can also be of any vertebrate (e.g., mouse, chicken, rabbit, goat or human), or of a mixture of vertebrates (e.g., humanized mouse).

Aptamers are single stranded oligonucleotides or oligonucleotide analogs that bind to a particular target molecule, such as a protein or a small molecule (e.g., a steroid or a drug, etc.). See discussion in PCT Patent Application Publication WO05/033257.

Additionally, the pancreatic islet β-cell in these methods can be treated ex vivo or in vitro (e.g., on islet β cells that are for transplanting into a patient having type 1 diabetes). In preferred embodiments, the islet β-cell is part of a pancreas of a mammal at risk for or having type 1 diabetes. While the methods are not limited to the use with any particular mammal, a mouse or a human is preferred.

In some embodiments of these methods, the β cell is also treated with an MHC class I molecule that is capable of binding the polypeptide. Preferably, the MHC class I molecule is an HLA-A2.

The MHC class I molecules in these embodiments is preferably in the form of a tetramer, as described above. In these embodiments, the CD8⁺ T cell binds the polypeptide-MHC combination, preventing the T cell from binding to the polypeptide-MHC on the β-cell.

In some embodiments of these methods, the polypeptide or MHC molecule further comprises a cytotoxic molecule, as described above, such that the T cell is killed when it binds to the polypeptide-MHC-cytotoxic molecule combination.

The pancreatic islet β-cell in these methods can also be treated ex vivo or in vitro (e.g., on islet p cells that are for transplanting into a patient having type 1 diabetes). In preferred embodiments, the islet β-cell is part of a pancreas of a mammal at risk for or having type 1 diabetes. While the methods are not limited to the use with any particular mammal, a mouse or a human is preferred.

In related embodiments, the invention is also directed to additional methods of preventing a CD8⁺ T cell that is cytotoxic to pancreatic islet β-cells from destroying a mammalian β-cell. The methods comprise treating the CD8⁺ T cell with a polypeptide 8-10 amino acids or analogs in length in a manner sufficient to reduce CD8+ T cells reactive to IGRP. In these embodiments, the polypeptide comprises one of the sequences (F/L)(G/N)IDLLWSV, VLFGLGFAI, or A(F/L)IPY(C/S)VHM, preferably FGIDLLWSV, LNIDLLWSV, VLFGLGFAI, ALLPYCVHM, or AFIPYSVHM. As with the similar embodiments discussed above, the polypeptide can further comprise a cytotoxic molecule.

These methods can also include treating the CD8⁺ T cell with a second polypeptide, where the second polypeptide targets an additional set of CD8⁺ T cells that are cytotoxic to pancreatic islet β-cells. This second target can, for example, be one of the other IGRP peptides that bind HLA-A2-restricted T cells, i.e., (F/L)(G/N)LDLLWSV, VLFGLGFAI, or A(F/L)IPY(C/S)VHM, preferably FGIDLLWSV, LNIDLLWSV, VLFGLGFAI, ALIPYCVHM, or AFIPYSVHM. The second target can also be other IGRP-derived peptides, for example YLKTN(A/I/L/V)FL, FLWSVFWLI, (T/A)YY(G/T)FLNFM, LR(L/V)(F/L)(G/N)IDLL, KWCANPDWI, and SFCKSASIP, as described in PCT Patent Publication WO05/033267. The second peptide can also be from an additional antigen targeted by cytotoxic CD8⁺ T cells, for example a peptide having one of the sequences XX(I/D/F/L)ENY(I/L)(E/W/Y)(L/M) or VMLENYTHL, as described in PCT Patent Publication WO06/023211.

As with related embodiments described above, the pancreatic islet β-cell can be part of a pancreas of a mammal at risk for or having type 1 diabetes.

In additional embodiments, the invention is directed to methods of treating a mammal that is at risk for type 1 diabetes. The methods comprise administering a polypeptide to the mammal in a manner sufficient to reduce CD8⁺ T cells reactive to IGRP. In these embodiments, the polypeptide is 8-10 amino acids or analogs in length, and comprises (F/L)(G/N)IDLLWSV, VLFGLGFAI, or A(F/L)IPY(C/S)VHM. Preferably, the polypeptide comprises FGIDLLWSV, LNIDLLWSV, VLFGLGFAI, ALIPYCVHM, or AFIPYSVHM.

Analogous to above-described embodiments, the polypeptide can further comprise a cytotoxic molecule. Also like those embodiments, these methods can further comprise treating the mammal with a second polypeptide 8-10 amino acids or analogs in length, e.g., comprising one of the sequences (F/L)(G/N)IDLLWSV, VLFGLGFAI, or A(F/L)IPY(C/S)VHM, one of the sequences YLKTN(A/I/L/V)FL, FLWSVFWLI, (T/A)YY(G/T)FLNFM, LR(L/V)(F/L)(G/N)IDLL, KWCANPDWI, or SFCKSASIP, or one of the sequences XX(I/D/F/L)ENY(I/L)(E/W/Y)(L/M) or VMLENYTHL.

In related embodiments, the invention is directed to additional methods of treating a mammal that is at risk for type 1 diabetes. The methods comprise administering a polypeptide to the mammal in a manner sufficient to reduce CD8⁺ T cells reactive to IGRP. In these embodiments, the polypeptide is 8-10 amino acids or analogs in length, completely homologous with a mammalian IGRP having at least 90% homology to SEQ ID NO: 1 or SEQ ED NO:2, and is capable of binding a human HLA-A2 MHC class I molecule

As discussed above in the context of other methods, the polypeptide can further comprise a cytotoxic molecule. Also as with those embodiments, these methods can further comprise treating the mammal with a second polypeptide 8-10 amino acids or analogs in length, e.g., comprising one of the sequences (F/L)(G/N)IDLLWSV, VLFGLGFAI, or A(F/L)EPY(C/S)VHM, one of the sequences YLKTN(A/I/L/V)FL, FLWSVFWLI, (T/A)YY(G/T)FLNFM, LR(L/V)(F/L)(G/N)EDLL, KWCANPDWI, or SFCKSASEP, or one of the sequences XX(I/D/F/L)ENY(I/L)(E/W/Y)(L/M) or VMLENYTHL.

The invention is also directed to methods of treating a mammal that has type 1 diabetes. The methods comprise administering a polypeptide to the mammal in a manner sufficient to treat diabetes, where the polypeptide is 8-10 amino acids or analogs in length, and comprises (F/L)(G/N)EDLLWSV, VLFGLGFAI, or A(F/L)EPY(C/S)VHM, preferably FGIDLLWSV, LNIDLLWSV, VLFGLGFAI, ALEPYCVHM, or AFEPYSVHM.

As discussed above in the context of other methods, the polypeptide can further comprise a cytotoxic molecule. Also as with those embodiments, these methods can further comprise treating the mammal with a second polypeptide 8-10 amino acids or analogs in length, e.g., comprising one of the sequences (F/L)(G/N)EDLLWSV, VLFGLGFAI, or A(F/L)IPY(C/S)VHM, one of the sequences YLKTN(A/I/L/V)FL, FLWSVFWLI, (T/A)YY(G/T)FLNFM, LR(L/V)(F/L)(G/N)IDLL, KWCANPDWI, or SFCKSASIP, or one of the sequences XX(I/D/F/L)ENY(I/L)(E/W/Y)(L/M) or VMLENYTHL.

In related embodiments, the invention is directed to additional methods of treating a mammal that has type 1 diabetes. The methods comprise administering a polypeptide to the mammal in a manner sufficient to treat type 1 diabetes, where the polypeptide is 8-10 amino acids or analogs in length, completely homologous with a mammalian IGRP having at least 90% homology to SEQ ED NO:1 or SEQ ED NO:2, and is capable of binding a human HLA-A2 MHC class I molecule

As discussed above in the context of other methods, the polypeptide can further comprise a cytotoxic molecule. Also as with those embodiments, these methods can further comprise treating the mammal with a second polypeptide 8-10 amino acids or analogs in length, e.g., comprising one of the sequences (F/L)(G/N)IDLLWSV, VLFGLGFAI, or A(F/L)IPY(C/S)VHM, one of the sequences YLKTN(A/I/L/V)FL, FLWSVFWLI, (T/A)YY(G/T)FLNFM, LR(L/V)(F/L)(G/N)IDLL, KWCANPDWI, or SFCKSASIP, or one of the sequences XX(I/D/F/L)ENY(I/L)(E/W/Y)(L/M) or VMLENYTHL.

Additionally, the invention is directed to kits comprising a sterile preparation comprising any of the above-described polypeptides, in a container.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### Identification of IGRP Peptides Recognized by Islet-Infiltrating T Cells in NOD Mice Expressing only HLA-A2

Studies utilizing the NOD mouse model of the disease have shown that the earliest initiation, as well as most of the progression, of beta cell destruction leading to disease requires contributions from major histocompatibility complex (MHC) class I-restricted beta cell-autoreactive CD8⁺ T cells. Insulitic lesions from newly diagnosed and graft-recurrent type 1 diabetes (T1D) patients also contain a prevalent population of CD8⁺ T cells. We describe here experimental data regarding IGRP reactivity to β cell-specific CD8⁺ T cells restricted to the common human class I molecule HLA-A2. Characterization of the HLA-A2-restricted response to beta cells could generate widespread benefit, because HLA-A2 is present in approximately 30-50% of members of many different ethnic groups and appears to be a diabetes susceptibility marker in certain populations. In order to identify the antigenic beta cell peptides recognized by HLA-A2-restricted T cells, we employed NOD mice transgenically expressing HLA-A2 in the absence of murine MHC class I molecules. Despite the expression of only a single MHC class I molecule, the HLA-A2-transgenic mice are diabetes-susceptible, with females showing a 50% incidence of disease at twenty-one weeks of age. We have cultured CD8⁺ T cells from the islets of the HLA-A2-transgenic NOD mice and have found that a subset of these T cells recognize the beta cell antigen islet-specific glucose-6-phosphatase catalytic subunit-related protein (IGRP), previously identified by our group as a target of H-2K^{d}-restricted T cells in standard NOD mice. We have mapped three novel epitopes of IGRP that are recognized by HLA-A2-restricted T cells in the transgenic NOD mice. Importantly, one of these antigenic peptides is conserved between mouse and human IGRP, and the other two are similar.

### Methods

Mice. All mice were housed under specific pathogen-free conditions. NOD/Lt mice are maintained at The Jackson Laboratory by brother-sister mating. Currently, diabetes develops in 90% of female and 63% of male NOD/Lt mice by 30 weeks of age. NOD.β2m^{null} (Serreze et al., 1994) and NOD.Rag1^{null} mice (Shultz et al., 2000) have been previously described. The HHD HLA-A2.1 transgene, kindly provided by Francois Lemonnier (Institut Pasteur, Paris, France), has been described (Pascolo et al., 1997). The transgene was injected directly into NOD zygotes, and founders carrying the HHD transgene were identified by PCR using forward primer 5′-CTTCATCGCAGTGGGCTAC-3′ and reverse primer 5′-CGGTGAGTCTGTGAGTGGG-3′. Cell surface expression of transgenic HLA-A2.1 molecules in NOD.HHD mice was confirmed by flow cytometry using fluorescein isothiocyanate (FITC)-conjugated mAb CR11-351 (Russo et al., 1983), kindly provided by Victor Engelhard (University of Virginia, Charlottesville). To eliminate expression of murine class I MHC molecules, the HHD transgene was crossed into the NOD.β2m^{null} stock, which led to development of the NOD.β2m^{null}.HHD strain.

Assessment of diabetes development. Diabetes development was defined by glycosuric values of ≧3 as assessed with Ames Diastix (kindly supplied by Miles Diagnostics, Elkhart, Ind.). Rates of diabetes development in the indicated experimental groups were assessed for statistically significant differences by Kaplan Meier life table analysis using the Statview 4.5 computer software program (Abacus Concepts, Berkeley, Calif.).

IGRP peptide library and individual synthetic peptides. A peptide library containing all of the 8 mer, 9 mer, 10 mer, and 11 mer peptides that can be derived from murine IGRP was synthesized by Mimotopes (Victoria, Australia) using their Truncated PepSet technology (Rodda, 2002). Each peptide mixture in the library contained four peptides with a common C-terminus, but having a length of 8, 9, 10, or 11 residues. The four peptides in each mixture were present in approximately equimolar amounts. A library of 348 peptide mixtures was synthesized to cover the 355 amino acids of the IGRP protein. Concentrated peptide stocks (2.75 mM) were prepared in 50% acetonitrile/H₂O, and 10 μM working stocks were obtained by serial dilution in PBS (pH 6.5). mIGRP₂₀₆₋₂₁₄ (VYLKTNVFL), mIGRP₂₂₆₋₂₃₆ (RLFGIDLLWSV), mIGRP₂₂₇₋₂₃₆ (LFGIDLLWSV), mIGRP₂₂₈₋₂₃₆ (FGIDLLWSV), mIGRP₂₂₉₋₂₃₆ (GIDLLWSV), mIGRP₂₆₅₋₂₇₃ (VLFGLGFAI), mIGRP₃₃₇₋₃₄₅ (ALIPYCVHM), hIGRP₂₂₈₋₂₃₆ (LNIDLLWSV), hIGRP₃₃₇₋₃₄₅ (AFIPYSVHM), PreproIAPP₅₋₁₃ (KLQVFLIVL), and Flu MP₅₈₋₆₆ (GILGFVFTL) peptides were synthesized by standard solid-phase methods using Fmoc chemistry in an automated peptide synthesizer (model 433A; Applied Biosystems, Foster City, Calif.), and their identities were confirmed by mass spectrometry. Concentrated stocks (10 mM) were prepared in DMSO, and 10 μM working stocks were obtained by dilution in PBS.

Pancreatic islet isolation and culture. Islet isolation by collagenase perfusion of the common bile duct was modified from a previously described protocol (Leiter, 1997). Briefly, the bile duct was cannulated and the pancreas perfused with collagenase P (Roche, Indianapolis, Ind.). The inflated pancreas was removed and incubated at 37° C. to digest exocrine tissue. Following dispersion of digested tissue and three washes with HBSS, islets were resuspended in HBSS containing DNase I (Worthington Biochemical, Lakewood, N.J.) and handpicked using a silanized micropipet under a dissecting microscope. Isolated islets were washed with 2% FBS in HBSS, resuspended in RPMI medium supplemented with 10% FBS (Hyclone, Logan, Utah) and 50 U/ml recombinant human IL-2 (PeproTech, Rocky Hill, N.J.), and cultured in 24-well tissue culture plates (˜50 islets/well) at 37° C., 5% CO₂ for 7 days.

IFN-γ ELISpot. ELISpot plates (MAHA S45 10; Millipore, Billerica, Mass.) were precoated with anti-murine IFN-γ Ab (R4-6A2; BD Biosciences Pharmingen, San Diego, Calif.) and blocked with 1% BSA (Fraction V, Sigma-Aldrich, St. Louis, Mo.) in PBS. APC (mitomycin C-treated T2 cells; ATCC, Manassas, Va.) were added at 2×10⁴ cells/well and pulsed with 1 μM peptide. Cultured islet-infiltrating T cells were added at 2×10⁴ cells/well and plates were incubated at 37° C. for 40 h. IFN-γ secretion was detected with a second, biotinylated anti-murine IFN-γ Ab (XMG1.2; BD Biosciences Pharmingen, San Diego, Calif.). Spots were developed using streptavidin-alkaline phosphatase (Zymed Laboratories, South San Francisco, Calif.) and 5-bromo-4-chloro-3-indolyl-phosphate/nitroblue tetrazolium chloride substrate (Sigma-Aldrich, St. Louis, Mo.) and counted using an automated ELISpot reader system (Autoimmun Diagnostika GmbH, Strassberg, Germany).

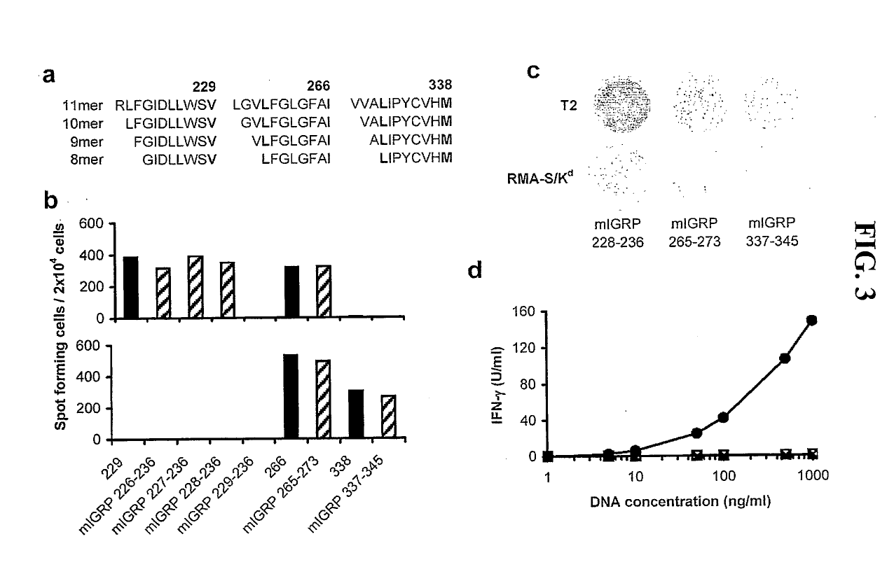

Transient transfection. COS-7 cells were transfected using a DEAE-dextran protocol as described (Karttunen et al., 1992). DNA (1 μg/ml) for the class I MHC molecule (HHD, H-2K^{d}, or H-2D^{b}) expression constructs was used along with varying concentrations of a murine IGRP expression construct or vector alone as indicated in the legend to FIG. 3d. Following co-culture with cultured islet-infiltrating T cells, T cell response was measured as IFN-γ release by ELISA using capture (R4-6A2) and detecting (biotinylated XMG1.2) anti-murine IFN-γ Abs purchased from BD Biosciences Pharmingen (San Diego, Calif.). Plates were developed with streptavidin-conjugated HRP (Southern Biotechnology Associates, Birmingham, Ala.) and tetramethyl benzidine (Pierce Biotechnology, Rockford, Ill.).

Peptide binding assay. Peptide binding to the HLA-A2 molecule was determined as described (Regner et al., 1996). Briefly, T2 cells were incubated for 18 hours at 26° C. and washed in serum-free culture medium. Cells (2×10⁵) in 60 μl serum-free medium were added to U-bottom 96-well culture plates with 20 μl peptide solution or PBS, and 20 μl of human β₂-microglobulin in PBS (final concentration 15 μg/ml, Sigma-Aldrich, St. Louis, Mo.). The cells were incubated for 20 hours at 37° C., 5% CO₂ in humidified air, then transferred into V-bottom 96-well culture plates and incubated in a water bath at 50° C. for 3 minutes. The cells were washed twice in cold (4° C.) PBS, stained with FITC-conjugated anti-HLA-A2 mAb BB7.2 (BD Biosciences Pharmingen, San Diego, Calif.), and analyzed by flow cytometry. The upregulation of HLA-A2 is reported as the fluorescence index (FI), defined as the mean fluorescence intensity (MFI) of the experimental sample divided by the MFI in the absence of peptide.

Cytotoxicity assays using intact islets as targets. Cytotoxicity assays using intact islets as targets were performed essentially as described (Choisy-Rossi et al., 2004). Briefly, human, NOD.HHD, and NOD.Rag1^{null} pancreatic islets (10 islets/well) were allowed to adhere in 96-well plates during a 13-17 day incubation at 37° C. in low-glucose DMEM medium. Adherent islets were then labeled with 5 μCi/well of ⁵¹Cr for 3 hours at 37° C. Islets were washed and overlaid with 100 μl of medium containing various numbers of cultured islet-infiltrating T cells from NOD.β2m^{null}. HHD mice. For establishing E:T ratios, each islet was assumed to contain ˜800 cells. A minimum of three wells were set up for each E:T ratio. Controls consisted of at least six wells of labeled NOD islets cultured in the absence of T cells. Following an overnight incubation at 37° C., the radioactivity in two fractions from each well was measured. The first fraction was the culture supernatant, and the second was obtained by solubilizing the remaining islets in 100 μl of 2% SDS. The % ⁵¹Cr release for each well was calculated by the formula [(supernatant cpm)/(supernatant cpm+SDS lysate cpm)]×100%. In turn, % specific cytotoxicity was calculated by subtracting the % ⁵¹Cr release from islets cultured in medium alone (i.e., spontaneous release) from the release by each well of islets cultured with a given number of T cells.

### Results

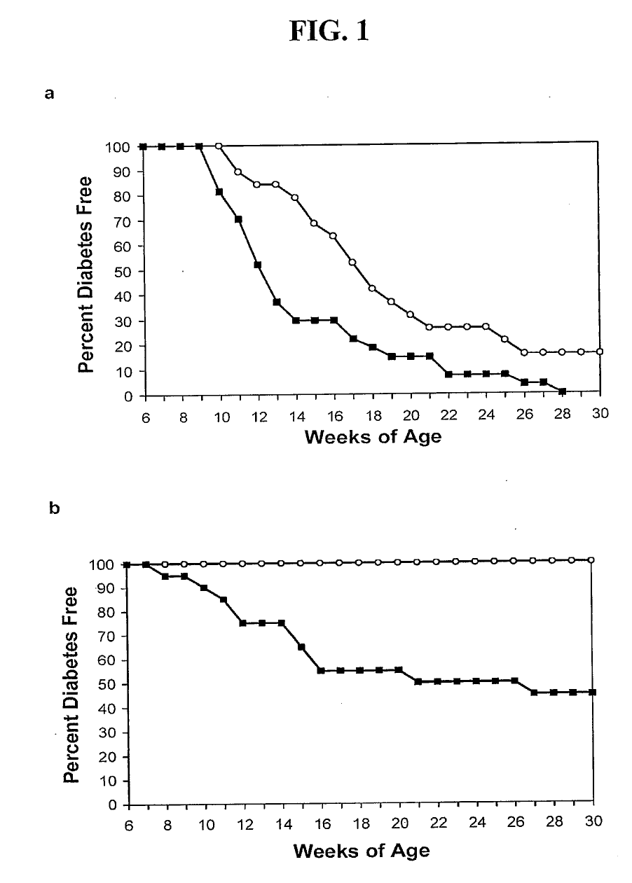

The transgenic mice having HLA-A2 as the only class I MHC molecule (NOD.β2m^{null}.HHD mice) had islet-infiltrating T cells that showed cytotoxic activity against HLA-A2-positive human pancreatic 0 cells (Table 1). Additionally, the NOD.β2m^{null}.HHD mice were diabetes-susceptible (FIG. 1).

TABLE 1
Cultured islet-infiltrating T cells from NOD.β2m^{null}.HHD
mice show cytotoxic activity against HLA-A2-positive
human pancreatic β cells

| | % specific cytotoxicity (% ± SEM) | | | |
|---|---|---|---|---|
| | HLA-A2^{a}/A68 | HLA-A11/A13 | | |
| E/T | human islet | human islet | NOD.HHD | NOD.Rag1^{null} |
| Ratio | cells | cells | islet cells | islet cells |
| 50:1^{b} | 21.8 ± 1.6 | 2.4 ± 1.1 | 18.4 ± 2.7 | 1.4 ± 0.7 |
| 10:1^{c} | 14.7 ± 0.5 | 2.8 ± 1.2 | 13.2 ± 3.1 | 0.6 ± 0.4 |
| 1:1^{c} | 16.1 ± 1.5 | 2.6 ± 2.3 | 13.6 ± 0.8 | 2.4 ± 0.8 |

Data represent the percentage of specific cytotoxicity exerted against the indicated target islet cells by cultured islet-infiltrating T cells from NOD.β2m^{null}.HHD mice.
⁵¹Cr release from islets cultured in medium alone (i.e., spontaneous release) for each target was measured in nine independent wells to calculate specific lysis as described in Methods.
^{a}HLA-A*0201
^{b}Six or
^{c}three independent wells were set up to calculate average and SEM.

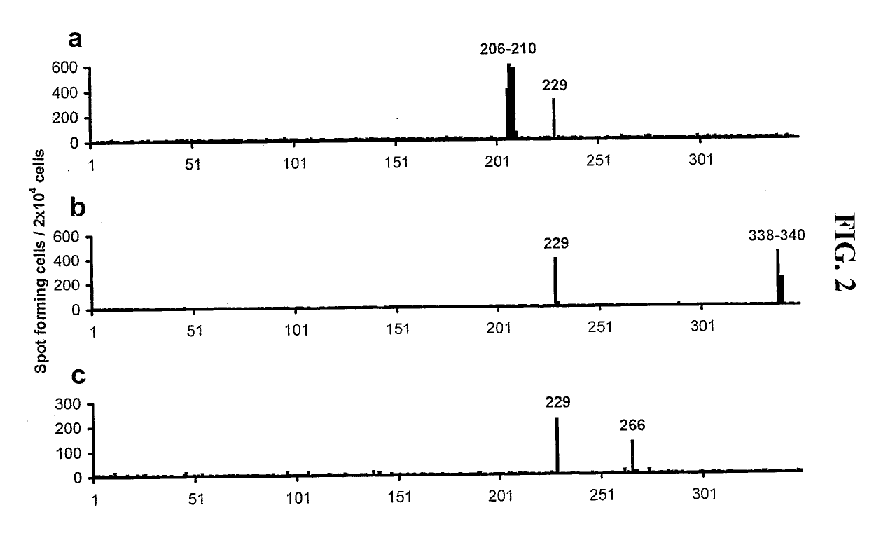

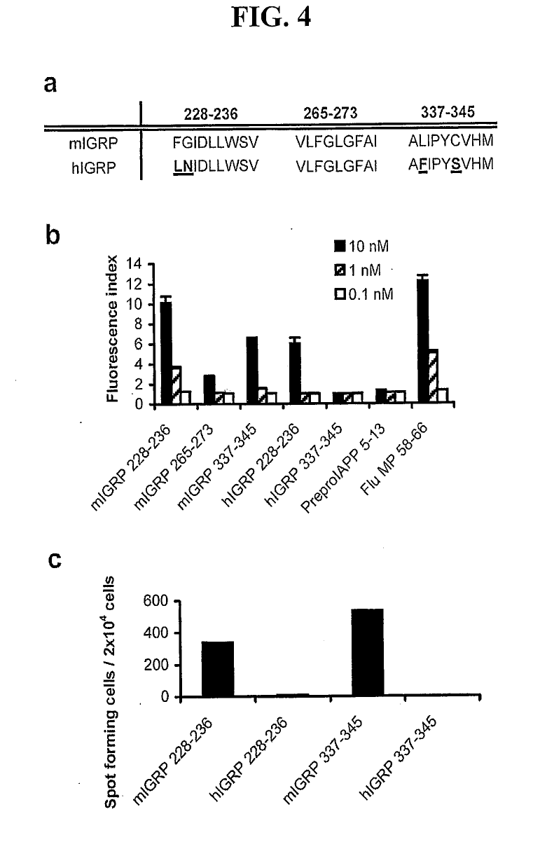

As shown in FIG. 2, cultured islet-infiltrating T cells from NOD.βB2m^{null}.HHD mice recognized three peptides not previously known to be targeted by cytotoxic T cells. FIG. 3 shows how the peptides were identified. The human counterparts to these peptides were identified as indicated in FIG. 4.

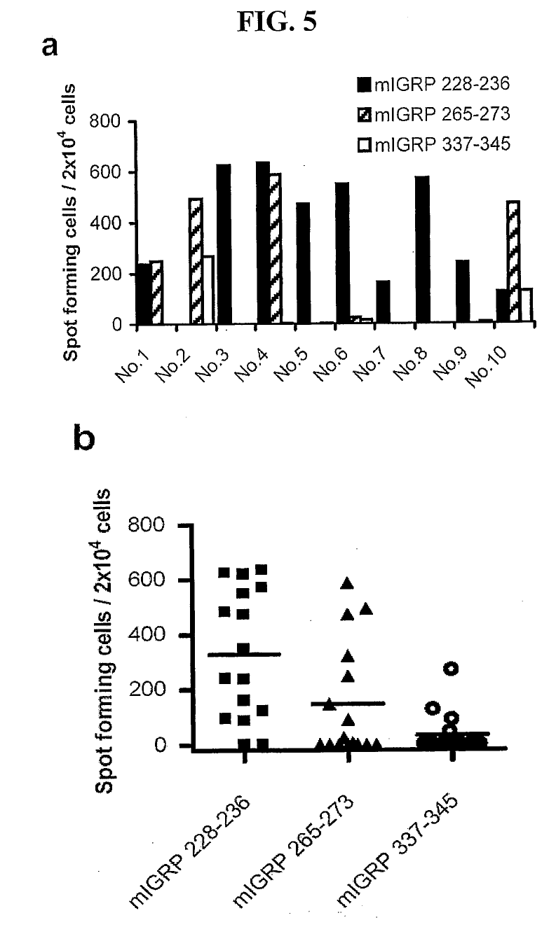

When individual mice were tested for CD8⁺ T cell reactivity to the three HLA-A2-binding IGRP peptides, variability in reactivity to the three peptides among the mice were noted (FIG. 5).

### EXAMPLE 2

### Further Characterization of IGRP Peptides Recognized by Islet-Infiltrating T Cells in NOD Mice Expressing Only HLA-A2

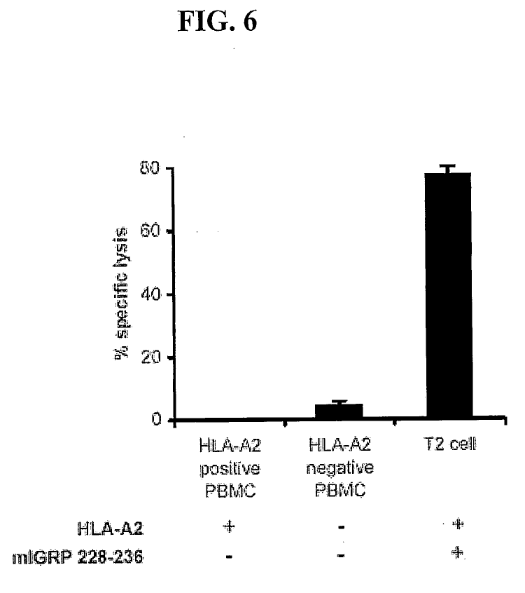

Table 1 of Example 1 showed that cultured islet-infiltrating T cells from NOD.β2m^{null}.HHD mice exhibit cytotoxic activity against HLA-A2-positive human islets. The islet specificity and HLA-A2 restriction was confirmed by showing that these same T cells were not able to recognize Con A blasts generated from HLA-A2-positive or HLA-A2-negative humans (FIG. 6).

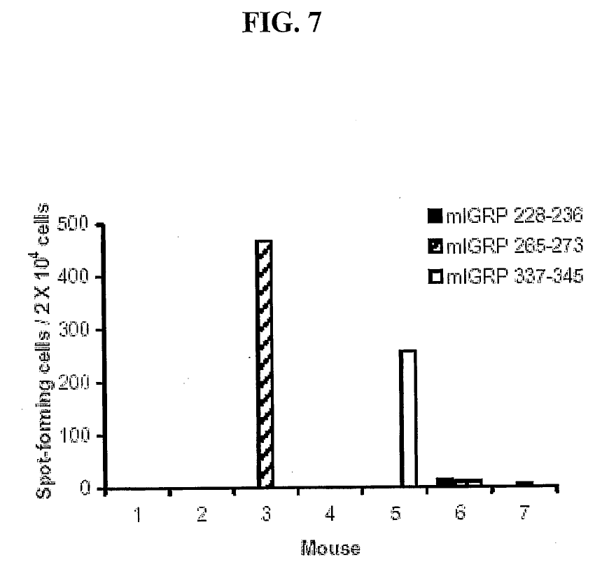

Example 1 showed that islet-infiltrating T cells from 12-wk-old NOD.β2m^{null}.HHD mice reacted to three IGRP peptides. To further demonstrate the importance of these IGRP peptides in disease development, T cells from the islets of very young (i.e., 5-wk-old) mice were cultured, representing the very early islet infiltrate. As shown in FIG. 7, each IGRP peptide was recognized by at least one of these young mice. Thus, reactivity to the identified IGRP peptides is one of the early markers in the elimination of β cells by HLA-A2-restricted T cells.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

### APPENDIX

### Mouse and Human IGRP Amino Acid Sequences

SEQ ID NO: 1—Mouse IGRP, from NP 067306. The peptides identified herein are underlined.

| | |
|---|---|
| 1 | mdflhrsgvl iihhlqedyr tyygflnfms nvgdprnifs iyfplwfqln qnvgtkmiwv |
| 61 | avigdwfnli fkwilfghrp ywwiqeteiy pnhsspcleq fpttcetgpg spsghamgss |
| 121 | cvwyvmvtaa lsytisrmee ssvtlhrltw sflwsvfwli qisvcisrvf iathfphqvi |
| 181 | lgviggmlva eafehtpgvh maslsvylkt nvflflfalg fylllrlfgi dllwsvpiak |
| 241 | kwcanpdwih idstpfaglv rnlgvlfglg fainsemflr scqgengtkp sfrllcalts |
| 301 | lttmqlyrfi kipthaeplf yllsfcksas iplmvvalip ycvhmlmrpg dkktk |

SEQ ID NO:2—Human IGRP, from NP 066999. The peptides identified herein are underlined.

| | |
|---|---|
| 1 | mdflhrngvl iiqhlqkdyr ayytflnfms nvgdprniff iyfplcfqfn qtvgtkmiwv |
| 61 | avigdwlnli fkwilfghrp ywwvqetqiy pnhsspcleq fpttcetgpg spsghamgas |
| 121 | cvwyvmvtaa lshtvcgmdk fsitlhrltw sflwsvfwli qisvcisrvf iathfphqvi |
| 181 | lgviggmlva eafehtpgiq taslgtylkt nlflflfavg fylllrvlni dllwsvpiak |
| 241 | kwcanpdwih idttpfaglv rnlgvlfglg fainsemfll scrggnnytl sfrllcalts |
| 301 | ltilqlyhfl qiptheehlf yvlsfcksas ipltvvafip ysvhmlmkqs gkksq |

1. An isolated and purified polypeptide comprising at least one of amino acid or analog sequences (F/L)(G/N)IDLLWSV (SEQ ID NO:3). VLFGLGFAI (SEQ ID NO:4), and A(F/L)IPY(C/S)VHM (SEQ ID NO:5), wherein the polypeptide does not comprise any of the sequences YLKTN(A/I/L/V)FL (SEQ ID NO:6). FLWSVFWLI (SEQ ID NO:7), (T/A)YY(G/T)FLNFM (SEQ ID NO:8). LR(L/V)(F/L)(G/N)IDLL (SEQ ID NO:9), KWCANPDWI (SEQ ID NO:10), or SFCKSASIP (SEQ ID NO:11).

2. The polypeptide of claim 1, completely homologous to a mammalian IGRP having at least 90% homology to SEQ ID NO:1 or SEQ ID NO:2.

3-4. (canceled)

5. The polypeptide of claim 1, consisting of 13-25 amino acids or analogs.

6. The polypeptide of claim 1, consisting of 8-10 amino acids or analogs.

7. The polypeptide of claim 1, comprising FGIDLLWSV (SEQ ID NO:12).

8. The polypeptide of claim 1, comprising LNIDLLWSV (SEQ ID NO:13).

9. The polypeptide of claim 1, comprising VLFGLGFAI (SEQ ID NO:4).

10. The polypeptide of claim 1, comprising ALIPYCVHM (SEQ ID NO:14).

11. The polypeptide of claim 1, comprising AFIPYSVHM (SEQ ID NO:15).

12. The polypeptide of claim 1, further comprising an antigenic carrier.

13. The polypeptide of claim 6, further comprising a detectable label.

14-16. (canceled)

17. The polypeptide of claim 13, further comprising an MHC class I molecule that is capable of binding the polypeptide.

18. The polypeptide of claim 6, further comprising an MHC class I molecule that is capable of binding the polypeptide.

19. (canceled)

20. The polypeptide of claim 18, further comprising a cytotoxic molecule.

21. (canceled)

22. The polypeptide of claim 5, further comprising an MHC class II molecule that is capable of binding the polypeptide.

23. The polypeptide of claim 22, further comprising a cytotoxic molecule.

24. The polypeptide of claim 1, in a sterile pharmaceutical preparation.

25. The polypeptide of claim 24, comprising at least one of the sequences FGIDLLWSV (SEQ ID NO:12). LNIDLLWSV (SEQ ID NO:13). VLFGLGFAI (SEQ ID NO:4), ALIPYCVHM (SEQ ID NO:14), or AFIPYSVHM (SEQ ID NO:15).

26-37. (canceled)

38. A method for determining whether a mammal is at risk for or has type 1 diabetes, the method comprising determining the presence of CD8⁺ T cells reactive to IGRP in the mammal by
a. obtaining a sample of lymphocytes comprising CD8⁺ T cells from the mammal;
b. combining the lymphocytes with a polypeptide and an MHC class I molecule that is capable of binding the polypeptide, wherein the polypeptide or the MHC molecule further comprises a detectable label and wherein the polypeptide is 8-10 amino acids or analogs in length and comprises one of the sequences (F/L)(G/N)IDLLWSV (SEQ ID NO:3), VLFGLGFAI (SEQ ID NO:4), or A(F/L)IPY(C/S)VHM (SEQ ID NO:5); and
c. determining whether any CD8⁺ T cells specifically bind to the polypeptide,
wherein CD8⁺ T cell binding to the polypeptide indicates that the mammal is at risk for or has type 1 diabetes.

39-66. (canceled)

67. A method of treating a mammal that is at risk for type 1 diabetes, the method comprising administering a polypeptide to the mammal in a manner sufficient to reduce CD8⁺ T cells reactive to IGRP, wherein the polypeptide is 8-10 amino acids or analogs in length, and comprises (F/L)(G/N)IDLLWSV (SEQ ID NO:3). VLFGLGFAI (SEQ ID NO:4), or A(F/L)IPY(C/S)VHM (SEQ ID NO:5).

68-89. (canceled)


SEQUENCE LISTING (32 sequences)

SEQ ID NO: 1 - protein, 355 aa, Mus musculus
MDFLHRSGVLIIHHLQEDYRTYYGFLNFMSNVGDPRNIFSIYFPLWFQLNQNVGTKMIWVAVIGDWFNLIFKWILFGHRPYWWIQETEIYPNHSSPCLEQFPTTCETGPGSPSGHAMGSSCVWYVMVTAALSYTISRMEESSVTLHRLTWSFLWSVFWLIQISVCISRVFIATHFPHQVILGVIGGMLVAEAFEHTPGVHMASLSVYLKTNVFLFLFALGFYLLLRLFGIDLLWSVPIAKKWCANPDWIHIDSTPFAGLVRNLGVLFGLGFAINSEMFLRSCQGENGTKPSFRLLCALTSLTTMQLYRFIKIPTHAEPLFYLLSFCKSASIPLMVVALIPYCVHMLMRPGDKKTK

SEQ ID NO: 2 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 3 - protein, 9 aa, Artificial
  consensus sequence
XXIDLLWSV

SEQ ID NO: 4 - protein, 9 aa, Homo sapiens
VLFGLGFAI

SEQ ID NO: 5 - protein, 9 aa, Artificial
  consensus sequence
AXIPYXVHM

SEQ ID NO: 6 - protein, 8 aa, Artificial
  consensus sequence
YLKTNXFL

SEQ ID NO: 7 - protein, 9 aa, Mus musculus
FLWSVFWLI

SEQ ID NO: 8 - protein, 9 aa, Artificial
  consensus sequence
XYYXFLNFM

SEQ ID NO: 9 - protein, 9 aa, Artificial
  consensus sequence
LRXXXIDLL

SEQ ID NO: 10 - protein, 9 aa, Mus musculus
KWCANPDWI

SEQ ID NO: 11 - protein, 9 aa, Mus musculus
SFCKSASIP

SEQ ID NO: 12 - protein, 9 aa, Mus musculus
FGIDLLWSV

SEQ ID NO: 13 - protein, 9 aa, Homo sapiens
LNIDLLWSV

SEQ ID NO: 14 - protein, 9 aa, Mus musculus
ALIPYCVHM

SEQ ID NO: 15 - protein, 9 aa, Homo sapiens
AFIPYSVHM

SEQ ID NO: 16 - protein, 9 aa, Artificial
  consensus sequence
XXXENYXXX

SEQ ID NO: 17 - protein, 9 aa, Mus musculus
VMLENYTHL

SEQ ID NO: 18 - DNA, 19 bp, Artificial
  primer
cttcatcgcagtgggctac

SEQ ID NO: 19 - DNA, 19 bp, Artificial
  primer
cggtgagtctgtgagtggg

SEQ ID NO: 20 - protein, 9 aa, Mus musculus
VYLKTNVFL

SEQ ID NO: 21 - protein, 11 aa, Mus musculus
RLFGIDLLWSV

SEQ ID NO: 22 - protein, 10 aa, Mus musculus
LFGIDLLWSV

SEQ ID NO: 23 - protein, 8 aa, Mus musculus
GIDLLWSV

SEQ ID NO: 24 - protein, 9 aa, Homo sapiens
KLQVFLIVL

SEQ ID NO: 25 - protein, 9 aa, Artificial
  control sequence
GILGFVFTL

SEQ ID NO: 26 - protein, 11 aa, Mus musculus
RLFGIDLLWSV

SEQ ID NO: 27 - protein, 11 aa, Mus musculus
LGVLFGLGFAI

SEQ ID NO: 28 - protein, 10 aa, Mus musculus
GVLFGLGFAI

SEQ ID NO: 29 - protein, 8 aa, Mus musculus
LFGLGFAI

SEQ ID NO: 30 - protein, 11 aa, Mus musculus
VVALIPYCVHM

SEQ ID NO: 31 - protein, 10 aa, Mus musculus
VALIPYCVHM

SEQ ID NO: 32 - protein, 8 aa, Mus musculus
LIPYCVHM